(19) United States
(12) Patent Application Publication
(10) Pub. No.: US 2005/0152992 A1
(43) Pub. Date: Jul. 14, 2005

(54) Antimicrobial surface preparation and method for producing the same

(76) Inventors: Loyal M. Johnson JR., Tucson, AZ (US); Patrick Ansel Thompson, Tucson, AZ (US)

Correspondence Address: Dahl & Osterloth, LLP;Suite 3405, 555 17th Street, Denver, CO, 80202-3937, US

(21) Appl. No.: 10/756,875

(22) Filed: Jan. 13, 2004

(51) Int. Cl. A61K 33/38

(52) U.S. Cl. 424/618

(57) ABSTRACT

The claimed invention involves a novel preparation comprising a dispersion of wax and silver particles that is effective for reducing bacteria on surfaces. The surface preparation according to the present invention comprises particles of silver dispersed in wax. A method for making the surface preparation according to the present invention includes combining a supply of wax with a supply of silver particles.

This invention relates to antimicrobial surface preparations in general and more specifically to an antimicrobial surface preparation comprising a dispersion of wax and silver particles and a method for producing a wax dispersion composition.

### BACKGROUND OF THE INVENTION

The use of silver and other metals, such as copper and aluminum, as disinfecting agents in water is well known. It is believed that metal ions may inactivate bacteria or viruses by interfering with cell function. It is also known that silver remains stable in solution and adsorbs to surfaces accounting for its continued germicidal effect over time.

The challenge has been to translate the disinfectant properties of metals in water to applications in which it is impractical or impossible to use them in water. In particular, a need for such disinfecting properties is great on many and varied surfaces in public places, such as hospitals and schools, where such surfaces are breeding grounds for bacteria, fungi and other harmful microorganisms.

Other approaches to solving the problem of combating the effects of harmful microorganisms on surfaces have taken the form of incorporating specific antimicrobial compounds, such as carboxylic acids and organotin compounds, into paints, for example. However, such compounds may prove toxic or unstable and are therefore of limited practical utility in hospitals, schools and other facilities where it is important to maintain as sterile an environment as practicable. Antibiotic ceramics have also been employed in fixed polymer coatings on a substrate, such as is disclosed in U.S. Pat. No. 6,436,422.

### SUMMARY OF THE INVENTION

The following summary is provided as a brief overview of the claimed product and process. It shall not limit the invention in any respect, with a detailed and fully-enabling disclosure being set forth in the Detailed Description of Preferred Embodiments section. Likewise, the invention shall not be restricted to any numerical parameters, processing equipment, chemical reagents, ingredients, operational conditions or other variables unless otherwise stated herein.

Embodiments of an antimicrobial surface preparation may comprise wax to which is added silver in amounts ranging from about 0.005% to about 0.3% by weight particles of silver to produce a dispersion.

Other embodiments of an antimicrobial surface preparation may comprise wax to which silver with particle sizes from about 5 nanometers to about 100 nanometers is added to form a dispersion.

Still more embodiments of an antimicrobial surface preparation may comprise wax to which silver particles are added such that said silver particles are available to be ionized in an amount sufficient to kill bacteria. Other embodiments may demonstrate that about 99% of bacteria have been killed with 24 hours of applying the dispersion of wax and silver particles to the treated surface.

Embodiments of a method for producing an antimicrobial surface preparation comprise providing supplies of wax and silver particles and combining them to produce a dispersion.

### DETAILED EMBODIMENTS OF THE INVENTION

Broadly described, embodiments of antimicrobial surface preparations according to the present invention may comprise particles of silver dispersed in a wax. The term “wax” as used herein is broadly defined to include one or more natural or synthetic waxes or wax analogs, including paraffin wax, montan wax, carnuba wax, beeswax, scale wax, ozokerite, Utah wax, microcrystalline wax such as plastic and tank bottom derived microcrystalline waxes, wax substitutes such as Fischer-Tropsch wax, polyaklylens such as polyethylene, polypropylene, including blends and copolymers of them. “Dispersion” as used herein is broadly encompasses systems wherein there is a relatively uniform distribution of silver particles in a medium such that said silver particles are dispersed in the medium as an emulsion, a colloidal suspension, a solute, or any combination of them.

The particles of silver may be present in an amount of about 0.005 wt. % to about 0.3 wt. %, and more preferably in an amount of at least about 0.005 wt. %. The silver material may comprise fine particles of silver having a mean particle size from about 5 nanometers to about 100 nanometers, and more preferably from about 10 nanometers to about 50 nanometers, as measured by a Horiba laser scattering analyzer.

The silver nanoparticles are produced from elemental silver and can be made in the size ranges specified herein by a variety of processes known in the art. In addition, silver nanoparticles of the sizes specified herein are commercially available from the Climax Molybdenum Company of Sahuarita, Ariz. Nanosilver is also commercially available from Argonide of Sanford, Fla.; Inframat® Advanced Material, LLC of Farmington, Conn.; and Sumitomo Electric, U.S.A. of New York, N.Y. The wax is commercially available in a variety of ready-made wax preparations, including those for floor care, furniture care and protection of other surfaces, such as Armstrong Excelon® floor wax.

The antimicrobial effect of silver particles is enhanced by increasing the amount of surface area of the silver particles that is available to come in contact with bacteria cells. It is generally well-known that bacterial microbes range in size from about 0.5 microns to about 3 microns. Thus, the size of the particles of silver may be such that the spacing between them occurs in that range, preferably with a spacing of about 1 micron between each particle. If the spacing between silver particles is smaller than the size of the harmful microbes, then the bacteria cells cannot escape coming in contact with the silver particles. As a result, with smaller sizes of nanoparticles, the spacings between them may be reduced, permitting lower effective concentrations of silver added to the wax medium. For example, with nanoparticles of silver of about 60 nanometers in size, the silver particles may be added to the wax medium at a concentration of about 0.00185 grams per cubic centimeter to be spaced about 1 micron apart. With silver nanoparticles of about 30 nanometers in size, which is preferred, the concentration of silver particles in the wax medium may be reduced to about 0.00025 grams per cubic centimeter to achieve the same spacing of about 1 micron between silver particles. Thus, it is believed that embodiments employing particles of silver from about 5 nanometers to about 100 nanometers will also be effective in at least the weight percentages such as are disclosed herein.

In a preferred embodiment of the present invention, silver nanoparticles with an average size of about 30 nanometers were combined with Armstrong Excelon® floor wax to form a wax dispersion of silver particles of about 0.005 wt. % The wax dispersion was applied in a layer to the surface of ceramic tile. It was then treated with staphylococcus aureus bacteria. After 4 hours, about 98.9% of the bacteria were reduced on average. After 24 hours, about 99.9% of the bacteria were eradicated.

Other embodiments may comprise combining silver nanoparticles with Armstrong Excelon® floor wax or any other wax to form a wax dispersion of silver particles of about 0.025% wt. %. Such wax dispersion may be used to treat surfaces, such as ceramic or linoleum tiles, other flooring materials or other surfaces, to kill bacteria or diminish its presence or retard its growth.

Other embodiments may comprise combining silver parties with Armstrong Excelon® floor wax or any other wax to form a wax dispersion of silver particles of about 0.3% wt. %. Still other embodiments may comprise combining silver nanoparticles with Armstrong Excelon® floor wax or any other wax to form a wax dispersion of silver particles of about 0.98% wt. %. Such wax dispersions may be used to treat surfaces, such as ceramic or linoleum tiles, other flooring materials or other surfaces, to kill bacteria or diminish its presence or retard its growth.

Additional embodiments have comprised starting with silver particles believed to be contaminated with phenolic resin, resulting in silver particles with an appearance that is brown in color. These particles will be referred to herein as “brown silver.” These particles were about 30 nanometers in average size and were combined with Armstrong Excelon® floor wax to form a wax dispersion of about 0.246 wt. %. This wax dispersion was applied to ceramic tiles and treated with staphylococcus aureus bacteria. Again, after four hours, there was a reduction in bacteria of about 91%; after 24 hours, sampling showed that about 99.9% of the bacteria had died and were not reproduced. Thus, the presence of what was believed to be phenolic resin did not impair the effectiveness of the brown silver as an antimicrobial agent in the wax dispersions to which it was added.

Other embodiments may comprise combining brown silver particles with Armstrong Excelon® floor wax or any other wax to form a wax dispersion of silver particles of about 0.3% wt. %. Still other embodiments may comprise combining brown silver nanoparticles with Armstrong Excelon® floor wax or any other wax to form a wax dispersion of silver particles of about 0.98% wt. %. Again, such wax dispersions may be used to treat surfaces, such as ceramic or linoleum tiles, other flooring materials or other surfaces, to kill bacteria or diminish its presence or retard its growth.

In order to provide further information regarding the invention, the following examples are provided. The examples present are not intended to limit the invention in any respect:

### EXAMPLES 1-3

In Examples 1-3 varying amounts of nanosilver particles were combined with Armstrong Excelon® floor wax to produce dispersions of about 0.025%, 0.098% and 0.246% by weight, respectively. The size of the nanosilver particles was about 30 nanometers on average. Three ceramic tiles each were coated with Examples 1-3. Three tiles were coated with blank wax (i.e., untreated wax) containing no silver particles. Three bare tiles were also included in the testing.

Approximately 6.0×10⁵ colony forming units (CFU) of Staphylococcus aureus bacteria were applied to all of the tiles involved in the testing as set forth above. The ceramic tiles were placed in humidity chambers and incubated at room temperature (23° C.). After incubation periods of one hour, four hours and 24 hours, one tile of each type was swabbed and the surviving S. aureus bacteria were enumerated.

TABLE 1
Survival of Staphylococcus aureus (in CFU)

| | | | 0.025% | | 0.246% |
|---|---|---|---|---|---|
| Exposure | | Blank | Ag | 0.098% Ag | Ag |
| time | Bare Tile | Wax | Example 1 | Example 2 | Example 3 |
| 1 Hour | 4.80 × 10⁵ | 1.22 × 10⁵ | 6.5 × 10⁴ | 2.9 × 10⁴ | <5.0 × 10⁰ |
| 4 Hours | 2.32 × 10⁵ | 1.24 × 10³ | 5.0 × 10¹ | 7.0 × 10¹ | 2.0 × 10¹ |
| 24 Hours | 1.7 × 10⁶ | <5.0 × 10⁰ | <5.0 × 10⁰ | <5.0 × 10⁰ | <5.0 × 10⁰ |

Examples 1-3 show significant reductions in the presence of bacteria after either one hour of exposure, especially in the case of Example 3, or four hours of exposure in the case of Examples 1-3. While all tiles coated with wax (either treated or untreated) showed antimicrobial effect after 24 hours, Examples 1-3 began to kill significant amounts of bacteria almost immediately within one hour, especially Example 3 which exhibited practically no bacteria after one hour.

### EXAMPLES 4-6

In Examples 4-6, nanosilver particles in varying amounts were combined with Armstrong Excelon® floor wax to produce dispersions of about 0.025%, 0.098%, and 0.246% by weight, respectively. The size of the nanosilver particles was about 30 nanometers on average. Nine ceramic tiles each were coated with the dispersions from Examples 4-6. Nine tiles were coated with blank wax (i.e., untreated wax) containing no silver particles. Nine bare tiles were also included in the testing.

Approximately 4.9×10⁶ CFU of Staphylococcus aureus bacteria were applied to all of the tiles involved in the testing as set forth above. The ceramic tiles were placed in humidity chambers and incubated at room temperature (23° C.). After incubation periods of one hour, four hours and 24 hours, three tiles of each type were swabbed and the surviving S. aureus bacteria were enumerated.

TABLE 2
Survival of Staphylococcus aureus (in CFU)

| | | | 0.025% | 0.098% | 0.246% |
|---|---|---|---|---|---|
| Exposure | | | Ag | Ag | Ag |
| time | Bare Tile | Blank Wax | Example 4 | Example 5 | Example 6 |
| 1 Hour | 1.2 × 10⁶ | 5.9 × 10⁵ | 2.3 × 10⁵ | 3.5 × 10⁵ | 3.1 × 10⁵ |
| | 7.6 × 10⁵ | 6.5 × 10⁵ | 2.2 × 10⁵ | 2.4 × 10⁵ | 2.1 × 10⁵ |
| | 7.2 × 10⁵ | 3.4 × 10⁵ | 2.3 × 10⁵ | 3.3 × 10⁵ | 1.8 × 10⁵ |
| 4 Hours | 1.5 × 10⁶ | 2.2 × 10⁵ | 1.5 × 10³ | 1.9 × 10³ | 2.7 × 10² |
| | 1.5 × 10⁶ | 1.1 × 10⁵ | 1.3 × 10³ | 2.4 × 10³ | 3.7 × 10² |
| | 1.8 × 10⁶ | 3.1 × 10⁵ | 1.6 × 10³ | 1.9 × 10³ | 4.2 × 10² |
| 24 Hours | 4.4 × 10⁷ | 1.0 × 10¹ | 5.0 × 10⁰ | <5.0 × 10⁰ | <5.0 × 10⁰ |
| | 4.5 × 10⁷ | 1.0 × 10¹ | 5.0 × 10⁰ | <5.0 × 10⁰ | <5.0 × 10⁰ |
| | 3.9 × 10⁷ | <5.0 × 10⁰ | 1.0 × 10¹ | <5.0 × 10⁰ | <5.0 × 10⁰ |

For statistical purposes, all values as reported in Table 2 were changed to log₁₀ as shown in Table 3. Values less than 10 were entered as 1.0 (log of 10). The results were analyzed by the analysis of variance (ANOVA) technique. ANOVA is widely used as a statistical technique to uncover the main and interaction effects of categorical independent variables on an interval dependent variable. With respect to tiles treated with Examples 4-6, the decrease in viable Staphylococcus was significant within one hour (P≦0.05) when compared to the survival of the bacteria on bare tiles. Examples 4-6 showed a marked reduction in bacteria after four hours as compared to tiles treated with blank wax.

TABLE 3
Survival of Staphylococcus aureus on Panels (in log₁₀)

| Exposure | | | 0.025% Ag | 0.098% Ag | 0.246% Ag |
|---|---|---|---|---|---|
| time | Bare Tile | Blank Wax | Example 4 | Example 5 | Example 6 |
| 1 Hour | 6.08 | 5.77 | 5.36 | 5.54 | 5.49 |
| | 5.88 | 5.81 | 5.34 | 5.38 | 5.32 |
| | 5.86 | 5.53 | 5.36 | 5.52 | 5.26 |
| | | P = 0.1025 | P = 0.00114 | P = 0.006 | P = 0.0041 |
| 4 Hours | 6.18 | 5.34 | 3.18 | 3.28 | 2.43 |
| | 6.18 | 5.04 | 3.11 | 3.38 | 2.57 |
| | 6.26 | 5.49 | 3.20 | 3.28 | 2.62 |
| | | P = 0.0025 | P = 1.5 × 10⁻⁷ | P = 2.8 × 10⁻⁷ | P = 5.2 × 10⁻⁷ |
| 24 Hours | 7.64 | 1.0 | 1.0 | 1.0 | 1.0 |
| | 7.65 | 1.0 | 1.0 | 1.0 | 1.0 |
| | 7.59 | 1.0 | 1.0 | 1.0 | 1.0 |
| | | P = 3.7 × 10⁻¹⁰ | P = 3.7 × 10⁻¹⁰ | P = 3.7 × 10⁻¹⁰ | P = 3.7 × 10⁻¹⁰ |

As seen in Tables 2 and 3, the percentage reduction in bacteria for Examples 4-6 was at least 99.9% after 4 hours. This reduction was improved after 24 hours.

### EXAMPLES 7-10

In Examples 7 and 8, nanosilver particles in varying amounts were combined with Armstrong Excelon® floor wax to produce dispersions of about 0.005%, and 0.246% by weight, respectively. In addition, in Examples 9 and 10, nanoparticles of brown silver (believed to be contaminated with phenolic resin) in varying amounts were combined with Armstrong Excelon® floor wax to produce dispersions of about 0.005%, and 0.246% by weight, respectively. The size of all the nanosilver particles used in Examples 7-10 was about 30 nanometers on average. Nine ceramic tiles each were coated with Examples 7-10. Nine tiles were coated with blank wax (i.e., untreated wax) containing no silver particles. Nine bare tiles were also included in the testing.

Approximately 3.65×10⁶ CFU of Staphylococcus aureus bacteria were applied to all of the tiles involved in the testing as set forth above. The ceramic tiles were placed in humidity chambers and incubated at room temperature (23° C.). After incubation periods of one hour, four hours and 24 hours, three tiles of each type were swabbed and the surviving S. aureus bacteria were enumerated.

TABLE 4
Survival of Staphylococcus aureus (in CFU)

| | | | Brown | Brown | | |
|---|---|---|---|---|---|---|
| Exp. | | | 0.005% Ag | 0.246% Ag | 0.005% Ag | 0.246% Ag |
| time | Bare Tile | Blank Wax | Example 7 | Example 8 | Example 9 | Example 10 |
| 1 | 1.32 × 10⁶ | 4.47 × 10⁵ | 5.49 × 10⁵ | 3.65 × 10⁵ | 1.41 × 10⁵ | 1.82 × 10⁵ |
| hour | 2.55 × 10⁶ | 7.01 × 10⁵ | 5.02 × 10⁵ | 3.57 × 10⁵ | 2.21 × 10⁵ | 1.86 × 10⁵ |
| | 1.40 × 10⁶ | 4.47 × 10⁵ | 5.71 × 10⁵ | 3.21 × 10⁵ | 1.82 × 10⁵ | 2.02 × 10⁵ |
| 4 | 3.50 × 10⁶ | 5.52 × 10⁵ | 3.56 × 10⁵ | 7.10 × 10⁴ | 5.65 × 10⁴ | 1.20 × 10⁴ |
| hours | 3.40 × 10⁶ | 5.17 × 10⁵ | 2.58 × 10⁵ | 4.35 × 10⁴ | 4.70 × 10⁴ | 3.25 × 10⁴ |
| | 5.10 × 10⁶ | 4.95 × 10⁵ | 5.52 × 10⁵ | 2.25 × 10⁵ | 1.05 × 10⁴ | 1.85 × 10⁴ |
| 24 | 4.97 × 10⁷ | 7.05 × 10⁴ | 1.45 × 10⁴ | 2.40 × 10² | 5.00 × 10⁰ | 5.00 × 10⁰ |
| hours | 3.86 × 10⁷ | 7.17 × 10⁴ | 1.95 × 10⁴ | <5.0 × 10⁰ | 5.00 × 10⁰ | 5.00 × 10⁰ |
| | 3.11 × 10⁷ | 7.15 × 10⁴ | 1.50 × 10² | 5.00 × 10¹ | 1.40 × 10² | 5.00 × 10⁰ |

For statistical purposes, all values were changed to log₁₀ as shown in Table 5. The were analyzed by analysis of variance (ANOVA).

TABLE 5
Survival of Staphylococcus aureus (in log₁₀)

| | | | Brown | Brown | | |
|---|---|---|---|---|---|---|
| Exp. | | | 0.005% Ag | 0.246% Ag | 0.005% Ag | 0.246% Ag |
| time | Bare Tile | Blank Wax | Example 7 | Example 8 | Example 9 | Example 10 |
| 1 | 6.12 | 5.65 | 5.74 | 5.56 | 5.15 | 5.26 |
| hour | 6.41 | 5.85 | 5.70 | 5.55 | 5.34 | 5.27 |
| | 6.15 | 5.65 | 5.76 | 5.51 | 5.26 | 5.31 |
| | | P = 0.010936 | P = 0.006245 | P = 0.001818 | P = 0.002262 | P = 0.001581 |
| 4 | 6.54 | 5.74 | 5.55 | 4.87 | 4.75 | 4.08 |
| hours | 6.53 | 5.71 | 5.41 | 4.64 | 4.67 | 4.51 |
| | 6.71 | 5.69 | 5.74 | 5.35 | 4.02 | 4.27 |
| | | P = 0.000127 | P = 0.000788 | P = 0.001647 | P = 0.001362 | P = 0.00011 |
| 24 | 7.70 | 4.85 | 4.16 | 2.38 | 0.70 | <0.70 |
| hours | 7.59 | 3.86 | 4.29 | <0.70 | 0.70 | <0.70 |
| | 7.49 | 4.85 | 2.18 | 1.70 | 2.15 | <0.70 |
| | | P = 0.000789 | P = 0.004106 | P = 0.000259 | P = 0.000221 | P = 3.37 × 10⁻⁸ |

Most effective in reducing the bacteria after one, four and 24 hours were (in descending order) Example 10, Example 9 and Example 8. After 24 hours, the rate of reduction was greater than 99.9%. Since the dispersion containing 0.246% brown silver (Example 8) was among the most effective as reducing bacteria, the presence of what was believed to be phenolic resin did not impair the effectiveness of the antimicrobial behavior of the silver nanoparticles in the dispersions tested. Since the dispersions containing silver nanoparticles (without phenolic resin contamination) (Examples 10 and 9) more effectively reduced bacteria than the dispersions containing brown silver (Examples 8 and 7), the antimicrobial effect was believed to be due to the presence of silver nanoparticles and not as a result of the phenolic resin contamination. There was no significant different in the antimicrobial effect of Example 7 and blank wax at any of the sampling times (one hour, four hours or 24 hours).

### Add Additional Examples Here

In conclusion, the claimed product and process collectively represent an important development in the field of antimicrobial surface preparations. The product and process discussed above are novel, distinctive, and highly beneficial from a technical and utilitarian standpoint. Having herein set forth preferred embodiments of the present invention, it is anticipated that suitable modifications can be made thereto which will nonetheless remain within the scope of the invention. The invention shall therefore be construed in accordance with the following claims:

1. An antimicrobial surface preparation, comprising a dispersion of wax and particles of silver in an antimicrobially effective amount.

2. The antimicrobial surface preparation of claim 1 wherein said particles of silver have a size between about 5 nanometers and about 100 nanometers on average.

3. The antimicrobial surface preparation of claim 1 wherein said particles of silver comprise at least about 0.005% by weight.

4. The antimicrobial surface preparation of claim 1 wherein said particles of silver comprise between about 0.005% by weight and about 0.3% by weight.

5. The antimicrobial surface preparation of claim 1 wherein said particles of silver comprise between about 0.005% by weight and about 0.25% by weight.

6. The antimicrobial surface preparation of claim 1 wherein said particles of silver are dispersed in said dispersion with an average spacing of no more than about 1.0 micron between said particles of silver.

7. The antimicrobial surface preparation of claim 1 wherein said particles of silver are dispersed in said dispersion with an average spacing of between about 0.5 microns and about 3.0 microns between said particles of silver.

8. The antimicrobial surface preparation of claim 1 wherein said particles of silver are dispersed in said dispersion with an average spacing of no more that about 3.0 microns between said particles of silver.

9. The antimicrobial surface preparation of claim 1 wherein said particles of silver are dispersed in said dispersion with an average spacing of at least about 0.5 microns between said particles of silver.

10. The antimicrobial surface preparation of claim 2 wherein said particles of silver comprise at least about 0.005% by weight.

11. The antimicrobial surface preparation of claim 2 wherein said particles of silver comprise between about 0.005% by weight and about 0.3% by weight.

12. The antimicrobial surface preparation of claim 2 wherein said particles of silver comprise between about 0.005% by weight and about 0.25% by weight.

13. The antimicrobial surface preparation of claim 2 wherein said particles of silver are dispersed in said dispersion with an average spacing of no more than about 1.0 micron between said particles of silver.

14. The antimicrobial surface preparation of claim 2 wherein said particles of silver are dispersed in said dispersion with an average spacing of between about 0.5 microns and about 3.0 microns between said particles of silver.

15. The antimicrobial surface preparation of claim 2 wherein said particles of silver are dispersed in said dispersion with an average spacing of no more that about 3.0 microns between said particles of silver.

16. The antimicrobial surface preparation of claim 2 wherein said particles of silver are dispersed in said dispersion with an average spacing of at least about 0.5 microns between said particles of silver.

17. An antimicrobial surface preparation, comprising a dispersion of wax and particles of silver wherein said particles of silver are available to be ionized in an amount sufficient to kill bacteria.

18. The antimicrobial surface preparation of claim 17 wherein said bacteria are killed at a rate of about 99% within about 24 hours after said dispersion of wax and particles of silver has been applied.

19. The antimicrobial surface preparation of claim 17 wherein said particles of silver have a size between about 5 nanometers and about 100 nanometers on average.

20. The antimicrobial surface preparation of claim 17 wherein said particles of silver comprise at least about 0.005% by weight.

21. The antimicrobial surface preparation of claim 17 wherein said particles of silver comprise between about 0.005% by weight and about 0.3% by weight.

22. The antimicrobial surface preparation of claim 17 wherein said particles of silver comprise between about 0.005% by weight and about 0.25% by weight.

23. The antimicrobial surface preparation of claim 17 wherein said particles of silver are dispersed in said dispersion with an average spacing of no more than about 1.0 micron between said particles of silver.

24. The antimicrobial surface preparation of claim 17 wherein said particles of silver are dispersed in said dispersion with an average spacing of between about 0.5 microns and about 3.0 microns between said particles of silver.

25. The antimicrobial surface preparation of claim 17 wherein said particles of silver are dispersed in said dispersion with an average spacing of no more that about 3.0 microns between said particles of silver.

26. The antimicrobial surface preparation of claim 17 wherein said particles of silver are dispersed in said dispersion with an average spacing of at least about 0.5 microns between said particles of silver.

27. The antimicrobial surface preparation of claim 19 wherein said particles of silver comprise at least about 0.005% by weight.

28. The antimicrobial surface preparation of claim 19 wherein said particles of silver comprise between about 0.005% by weight and about 0.3% by weight.

29. The antimicrobial surface preparation of claim 19 wherein said particles of silver comprise between about 0.005% by weight and about 0.25% by weight.

30. The antimicrobial surface preparation of claim 19 wherein said particles of silver are dispersed in said dispersion with an average spacing of no more than about 1.0 micron between said particles of silver.

31. The antimicrobial surface preparation of claim 19 wherein said particles of silver are dispersed in said dispersion with an average spacing of between about 0.5 microns and about 3.0 microns between said particles of silver.

32. The antimicrobial surface preparation of claim 19 wherein said particles of silver are dispersed in said dispersion with an average spacing of no more that about 3.0 microns between said particles of silver.

33. The antimicrobial surface preparation of claim 19 wherein said particles of silver are dispersed in said dispersion with an average spacing of at least about 0.5 microns between said particles of silver.

34. A surface preparation, comprising a dispersion of wax and silver wherein said particles of silver comprise at least about 0.005% by weight.

35. The surface preparation of claim 34 wherein said particles of silver further comprise not more that about 0.3% by weight.

36. The surface preparation of claim 34 wherein said particles of silver further comprise not more than about 0.25% by weight.

37. The surface preparation of claim 34 wherein said particles of silver have a size between about 5 nanometers and about 100 nanometers on average.

38. A surface preparation, comprising a dispersion of wax and particles of silver wherein said particles of silver have a size between about 5 nanometers and about 100 nanometers on average.

39. A method for producing a surface preparation comprising:
providing a supply of wax;
providing a supply of particles of silver;
combining said supply of wax with said supply of particles of silver to form a dispersion comprising said wax and said particles of silver.

40. The method of claim 39 wherein said particles of silver have a size between about 5 nanometers and about 100 nanometers on average.

41. The method of claim 39 wherein said particles of silver comprise at least about 0.005% by weight.

42. The method of claim 39 wherein said particles of silver comprise between about 0.005% by weight and about 0.3% by weight.

43. The method of claim 39 wherein said particles of silver comprise between about 0.005% by weight and about 0.25% by weight.

44. The method of claim 39 wherein said particles of silver are dispersed in said dispersion with an average spacing of no more than about 1.0 micron between said particles of silver.

45. The method of claim 39 wherein said particles of silver are dispersed in said dispersion with an average spacing of between about 0.5 microns and about 3.0 microns between said particles of silver.

46. The method of claim 39 wherein said particles of silver are dispersed in said dispersion with an average spacing of no more than about 3.0 microns between said particles of silver.

47. The method of claim 39 wherein said particles of silver are dispersed in said dispersion with an average spacing of at least about 0.5 microns between said particles of silver.

48. The method of claim 39 wherein said particles of silver are dispersed in said dispersion in an antimicrobially effective amount.

49. A method for producing an antimicrobial surface preparation comprising:
providing a supply of Armstrong Excelon floor wax;
providing a supply of particles of silver wherein the size of each said particle of silver is about 30 nanometers on average;
combining said supply of Armstrong Excelon floor wax and said supply of particles of silver to form a dispersion comprising said wax and said particles of silver wherein said particles of silver comprise about 0.005% by weight and wherein said dispersion kills bacteria at a rate of about 99% within about 24 hours of its application.
